(19) United States
(12) Patent Application Publication
(10) Pub. No.: US 2025/0185981 A1
(43) Pub. Date: Jun. 12, 2025

(54) PRECISE, PORTABLE, NON-INVASIVE MELANOMA DETECTION DEVICE USING IMAGE-BASED DEEP LEARNING APPROACH

(71) Applicant: Shubhan Bhattacharya, Cary, NC (US)

(72) Inventor: Shubhan Bhattacharya, Cary, NC (US)

(21) Appl. No.: 18/974,138

(22) Filed: Dec. 9, 2024

Related U.S. Application Data
(60) Provisional application No. 63/607,154, filed on Dec. 7, 2023.

(51) Int. Cl. A61B 5/00; G06T 7/00; G16H 30/20; G16H 50/20

(52) U.S. Cl. CPC A61B 5/444; G06T 7/0012; G16H 30/20; G16H 50/20; G06T 2207/30088

(57) ABSTRACT

Various examples are provided related to detection of melanoma using an image-based deep learning approach. In one example, a portable melanoma detection device includes an imaging device and a processing or computing device. The processing or computing device can receive an image of a skin blemish captured by the imaging device; analyze the image to determine a condition of the skin blemish, the condition indicating whether the skin blemish is malignant or benign; and render the image for display in a user interface. The image can be displayed with the condition of the skin blemish. In another example, a method for melanoma detection includes acquiring an image of a skin blemish captured by an imaging device; analyzing the image to determine a condition of the skin blemish, the condition indicating whether the skin blemish is malignant or benign; and rendering the image for display in a user interface.

### CROSS REFERENCE TO RELATED APPLICATIONS

This application claims priority to, and the benefit of, U.S. provisional application entitled “Precise, Portable, Non-Invasive Melanoma Detection Device Using Image-Based Deep Learning Approach” having Ser. No. 63/607,154, filed Dec. 7, 2024, which is hereby incorporated by reference in its entirety.

### BACKGROUND

Melanoma is the most common of all skin cancers and is one of the fastest-growing cancers worldwide. Malignant Melanoma (MM) has the greatest mortality rate and highest dissemination potential. MM can be cured if detected early but left untreated, it poses a significant risk as it can metastasize to other organs. The prognosis of patients with metastatic MM is grim, with a 5-years survival rate between 5-19%. MM can go undetected in many impoverished rural areas in the US and worldwide, as many people in the United States and the world do not have easy access to a dermatologist. The American Cancer Society estimated 106,110 new melanoma cases with 7,180 deaths due to melanoma in the US in 2021. Health-care inequality is a real and fundamental global issue.

### SUMMARY

Aspects of the present disclosure are related to detection of melanoma using an image-based deep learning approach. In one example, among others, a portable melanoma detection device comprises an imaging device and a processing or computing device communicatively coupled with the imaging device. The processing or computing device is configured to: receive one or more image of a skin blemish captured by the imaging device; analyze the one or more image to determine a condition of the skin blemish, the condition indicating whether the skin blemish is malignant or benign; and render the one or more image for display in a user interface of the processing or computing device, the one or more image displayed with the condition of the skin blemish. In one or more aspects, analysis of the one or more image can be implemented by a deep learning classifier trained to determine the condition and a confidence number associated with the determined condition. A malignant condition can be indicated by a confidence number that is greater than 5. The deep learning classifier can determine the condition based upon averaging of analysis of a plurality of transformed versions of the one or more image. The one or more image can be flipped, transposed, or both to generate the plurality of transformed versions. Averaging of the analysis can comprise averaging an output of the deep learning classifier associated with analysis of each of the plurality of transformed versions. In various aspects, acquisition of the one or more image can be controlled through the user interface of the processing or computing device. The one or more image can be acquired directly from the imaging device or from memory of the processing or computing device. The one or more image can be acquired from memory of the processing or computing device. The skin blemish can be a mole.

In another aspect, a method for melanoma detection, comprises acquiring an image of a skin blemish captured by an imaging device of a portable melanoma detection device; analyzing, by a processing or computing device of the portable melanoma detection device, the image to determine a condition of the skin blemish, the condition indicating whether the skin blemish is malignant or benign; and rendering, by a processing or computing device, the image for display in a user interface of the portable melanoma detection device, the one or more image displayed with the condition of the skin blemish. In one or more aspects, the method can comprise determining that the skin blemish is a malignant melanoma and identifying a treatment for the malignant melanoma. The treatment can comprise removal of the malignant melanoma. In various aspects, analysis of the image can be implemented by a deep learning classifier trained to determine the condition based upon a confidence number. The analysis of the image can comprise generating a plurality of transformed versions by flipping the image, transposing the image, or both; analyzing each of the plurality of transformed versions to determine corresponding confidence numbers; and determining the condition of the skin blemish based upon an average of the corresponding confidence numbers. The confidence number can be an output of the deep learning classifier. The confidence number can be in a range from 1 to 10. The method can comprise capturing the image with the imaging device of the portable melanoma detection device; and storing the image in memory of the portable melanoma detection device, where the image is acquired from the memory. The method can comprise capturing the image with the imaging device of the portable melanoma detection device, wherein the image is acquired directly from the imaging device after capture. The skin blemish can be a mole.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a melanoma detection device design, in accordance with various embodiments of the present disclosure.

FIG. 2 illustrates an example of a melanoma detection device comprising an imaging device, a processing or computing device and a power supply, in accordance with various embodiments of the present disclosure.

FIG. 3 includes images of a prototype device for melanoma detection, in accordance with various embodiments of the present disclosure.

FIG. 4 illustrates an example of experimental results of the prototype device, in accordance with various embodiments of the present disclosure.

FIG. 5 illustrates an example of an output display of the prototype device, in accordance with various embodiments of the present disclosure.

FIG. 6 is a schematic block diagram of a processing or computing device, in accordance with various embodiments of the present disclosure.

### DETAILED DESCRIPTION

Disclosed herein are various examples related to detection of melanoma using an image-based deep learning approach. Reference will now be made in detail to the description of the embodiments as illustrated in the drawings, wherein like reference numbers indicate like parts throughout the several views.

Melanomas have a 99% cure rate if detected in the earliest stages. This fact motivated the development of a portable and precise Melanoma detection device that can take an image of a mole and conclude if it is “Malignant” or “Benign”. This device allows people in rural/impoverished areas where dermatologists are not available to have a timely detection of MM, thus leading to a better prognosis. This device will have the ability to be used as a preliminary diagnostic tool due to its low-cost, easy-to-use, non-invasive, and efficient approach to detecting MM. Timely detection may allow in-time treatment and can impact the health of millions worldwide. After the preliminary detection, the presence of a MM can be verified by a physician or other health practitioner and appropriate treatment (e.g., surgical removal) can be carried out.

### Detection Device

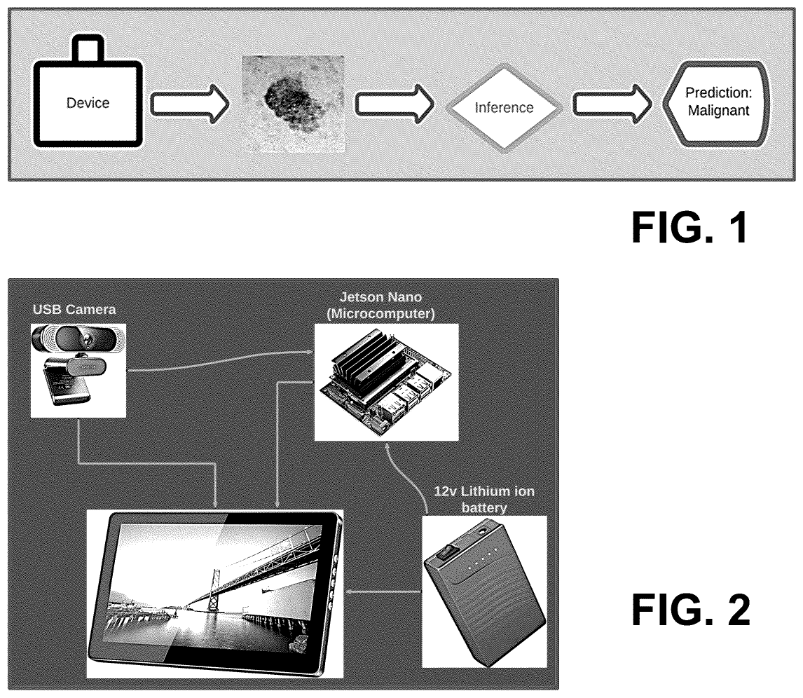

Referring to FIG. 1, shown is an example of a design of a Melanoma detection device. The portable device comprises an integrated camera that can take a picture of a skin lesion and provide a prediction in “real-time” whether the mole is “Malignant” or “Benign.”

The Melanoma detection is based on a deep learning model to classify skin lesions as either “Malignant” or “Benign”. A machine learning model trained on annotated Melanoma data can be used in order to actually detect Melanoma from an image. An open-source deep learning model suitable for this classification was researched and identified. The “SIIM-ISIC Melanoma Classification” model was selected. This model based on “Effnet-B7 w/ input size 640” was chosen due to its extremely high accuracy of 97% and the fact that it needed no metadata. The open source model was trained by others on the SIIM-ISIC dataset with the 97% accuracy. The SIIM-ISIC is a dataset that contains 33,126 dermoscopic training images of unique benign and malignant skin lesions from over 2,000 patients. All malignant diagnoses had been confirmed via histopathology, and benign diagnoses have been confirmed using either expert agreement, longitudinal follow-up, or histopathology. This model does not require any metadata and simply looks at an image to discern if it is Melanoma or a Benign mole. The input to the model is the image to be analyzed. The model can output a number in a range between 1-10, with 1 being a confident benign image and 10 being a confident malignant melanoma image. If an integer scale of 1-10 is the output, then 1-5 can indicate a benign mole with 1 being a confident indication and 5 being a benign but less confident indication. While 6-10 indicates a malignant melanoma with 10 being a confident melanoma output while 6 indicates a less confident melanoma output.

An intelligent inference engine using the deployed model was then developed and coded. The inference engine created was coded in Python, where a single image can be loaded and then run through the model a number of times (e.g., 8 times). The image was transformed (by flipping and/or transposing) differently each time it was run through the inference engine. The model for classification was trained using data provided by the International Skin Imaging Collaboration (ISIC) Archive, which is a publicly available collection of images of skin lesions. Common image augmentations from, e.g., the Pytorch augmentation library can be used. This ensured that the final prediction was the most accurate. The model and the inference engine were then extensively trained using large datasets of thousands of mole images obtained from open sources.

After this, the inference engine can be deployed with the model where after a photo of a mole is taken it is transformed in different ways, through simple reflections, etc. and then analyzed multiple times through the model to ensure a confident final prediction. For example, a photo can be taken and then transformed (by flipping and/or transposing) in, e.g., 4 different ways and the resulting images can be run through the model each time and the results can be averaged to ensure that the model confidently outputs a result on the data.

To develop a portable device, a microcomputer suitable for running the model and the inference engine had to be selected. Initially, Raspberry Pi 3 Model B was chosen, and it was determined that it did not have the capabilities of running this program. Then NVIDIA Jetson Nano 4 GB single board computer (SBC) was selected with Quad-core ARM A57@1.43 GHz CPU, where a camera could then be connected to it. The Jetson Nano also has a 4 GB RAM compared to the Raspberry Pi 3 Model B, which has 1 GB RAM. For the efficiency of the portable device, it was determined to use the CPU rather than the GPU. The program with the developed inference engine tested on a desktop computer was revised for the Jetson Nano using the CPU. The original model with the inference engine was trained using a GPU, and therefore, there were errors when running the inference program without the modifications on the Jetson Nano CPU. The deep learning tensors had to be dynamically remapped to the CPU to run correctly on the Jetson Nano.

### Prototype Device

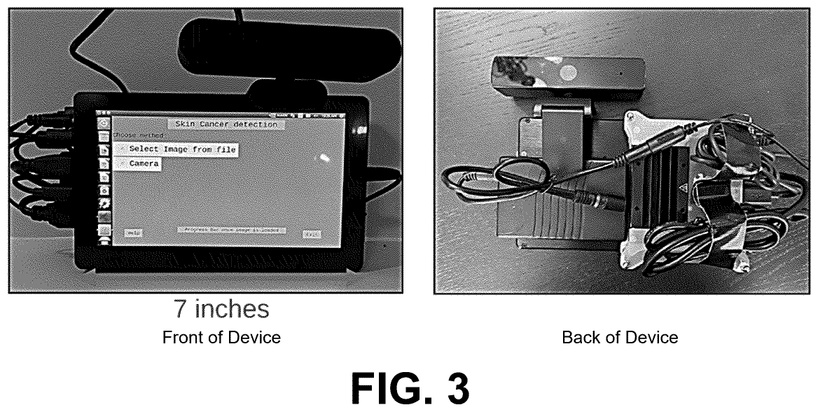

The developed device is a small tablet with a camera that runs on a Jetson Nano 4 GB and utilizes deep learning and an optimized inference system for diagnosis. The integration of the different subsystems of the device (e.g., NVIDIA Jetson Nano, USB Camera, 7″ Touchscreen Monitor, and 12V Li-Ion Battery) is shown in FIG. 2. A program was coded to incorporate a USB camera to take a picture and run it through the same inference engine. A touchscreen 7-inch monitor and a portable battery were integrated to complete the device. A 12V DC Lithium-ion battery was used to power both the device and the touchscreen monitor. The system was then tested against a few images online to ensure that the quality of a USB camera does not affect the model's accuracy. A Tkinter Graphical user interface [GUI] was created so that any user can take a picture of a skin lesion, which then begins the inference process and ends in a prediction. The final prototype device shown in FIG. 3 is 7×2 inches and weighs 250 g approximately.

### Experimental Results

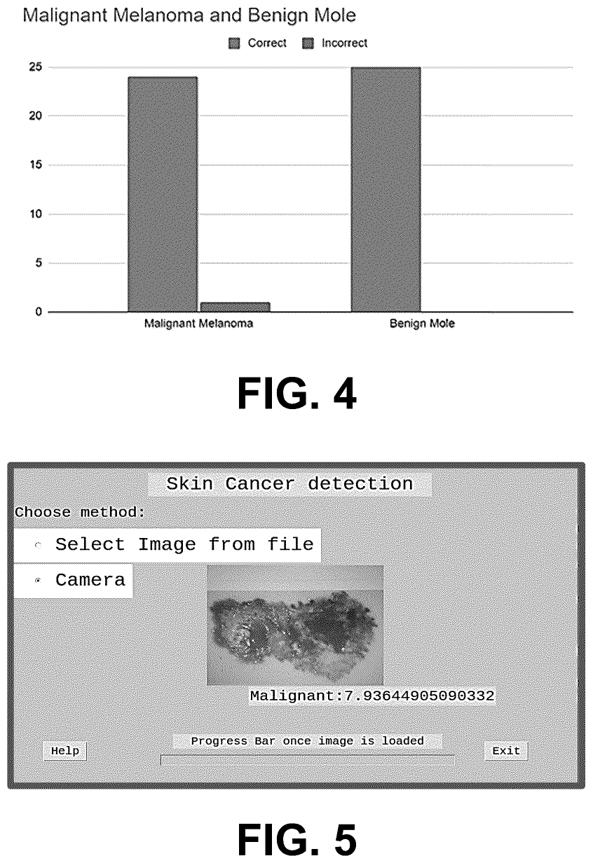

For the blinded experimental results, 50 images were obtained from online resources such as, e.g., Mayo Clinic, Skin Cancer Foundation, NHS, Medscape, Dermnet NZ, Cleveland Clinic, Very Well Health, and Medicine Net. They formed a test dataset as illustrated in FIG. 4. Of those images, 25 were Malignant Melanoma images, while the other 25 were Benign Moles. The device can also output a confidence indication (which can be, e.g., >5 for “Malignant” and ≤5 for “Benign” in a scale from 1-10). Samples were randomly numbered, and a user with no previous knowledge ran the samples through the device. Once the device completed a sample, the prediction and the confidence number were tabulated. After all 50 images were tested, the results were compared with the actual results from the dataset, and an accuracy of 98% was achieved. The device predicted only one malignant sample incorrectly, as shown in FIG. 4.

An example of the screen output of the prediction is shown in FIG. 5 for a sample that is “Malignant” with a confidence number of 7.9, indicating a very high confidence. The average run time is 4 min 45 seconds, with 1 minute of setup time. The battery life is 3 hours which allows for 30 predictions per charge. The prototype was tested against multiple images from open sources.

Dermatologists rely on visual inspection and, therefore, according to some studies, can typically accurately detect 86.6% of skin cancers from images. The prototype device has been demonstrated to achieve 98% accuracy based on blinded experimental results. It uses deep learning and an inference engine as the source of detection and does not need high-cost sensors. The single device cost is estimated to cost significantly less than $300 in volume production. The detection device can potentially impact the health of millions around the world due to its low-cost, easy-to-use, non-invasive, and efficient approach to detecting MM. Judiciously used, it can be widely distributed in community clinics which will help in timely detection of Melanoma, leading to an effective and life-saving prognosis. Furthermore, providers with minimal training can use this device.

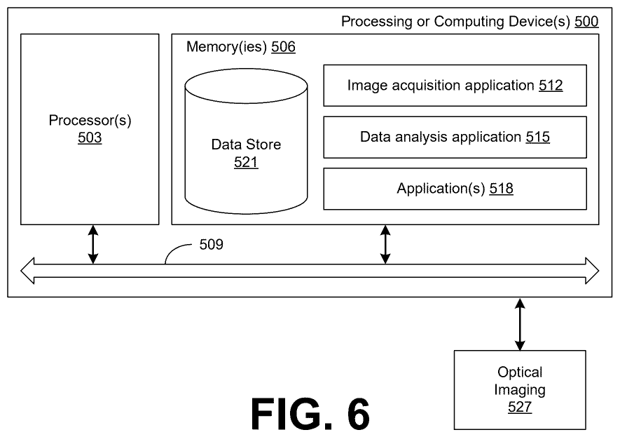

With reference now to FIG. 6, shown is a schematic block diagram of a computing device 500 according to an embodiment of the present disclosure. The computing device 500 includes at least one processor circuit, for example, having a processor 503 and a memory 506, both of which are coupled to a local interface 509. To this end, the computing device 500 may comprise, for example, at least one server computer or like device. The local interface 509 may comprise, for example, a data bus with an accompanying address/control bus or other bus structure as can be appreciated.

Stored in the memory 506 are both data and several components that are executable by the processor 503. In particular, stored in the memory 506 and executable by the processor 503 are an image acquisition application 512, a data analysis application 515, and potentially other applications 518. The image acquisition application 512 and/or the data analysis application 515 can implement, when executed by the computing device 500, various aspects of the computational processing as described above. For example, the image acquisition application 512 can facilitate acquisition and/or storage of acquired optical images and the data analysis application 515 can facilitate processing of the data. In some implementations, the image acquisition application 512 and data analysis application 515 may be combined in a single application. Also stored in the memory 506 may be a data store 521 including, e.g., recordings, images, video and other data. In addition, an operating system may be stored in the memory 506 and executable by the processor 503. It is understood that there may be other applications that are stored in the memory and are executable by the processor 503 as can be appreciated.

An optical imaging device 527 (e.g., an optical camera) in communication with the computing device 500 can be utilized to obtain optical images. Synchronization of each optical image can be coordinated by the image acquisition application 512. For example, the image acquisition application 512 can provide a control signal to initiate acquisition of the optical image to optical imaging device 527. The acquired optical image can be stored in memory by the image acquisition application 512 with a time stamp and/or frame number. After capturing and storing the data, the data analysis application 515 can produce the movie or series of frames including the optical information.

Where any component discussed herein is implemented in the form of software, any one of a number of programming languages may be employed such as, for example, C, C++, C#, Objective C, Java®, JavaScript®, Perl, PHP, Visual Basic®, Python®, Ruby, Delphi®, Flash®, or other programming languages. A number of software components are stored in the memory and are executable by the processor 503. In this respect, the term “executable” means a program file that is in a form that can ultimately be run by the processor 503. Examples of executable programs may be, for example, a compiled program that can be translated into machine code in a format that can be loaded into a random access portion of the memory 506 and run by the processor 503, source code that may be expressed in proper format such as object code that is capable of being loaded into a random access portion of the memory 506 and executed by the processor 503, or source code that may be interpreted by another executable program to generate instructions in a random access portion of the memory 506 to be executed by the processor 503, etc. An executable program may be stored in any portion or component of the memory including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 506 is defined herein as including both volatile and nonvolatile memory and data storage components. Volatile components are those that do not retain data values upon loss of power. Nonvolatile components are those that retain data upon a loss of power. Thus, the memory 506 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 503 may represent multiple processors 503 and the memory 506 may represent multiple memories 506 that operate in parallel processing circuits, respectively. In such a case, the local interface 509 may be an appropriate network that facilitates communication between any two of the multiple processors 503, between any processor 503 and any of the memories 506, or between any two of the memories 506, etc. The processor 503 may be of electrical or of some other available construction.

Although portions of the image acquisition application 512, data analysis application 515, and other various systems described herein may be embodied in software or code executed by general purpose hardware, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

The image acquisition application 512 and data analysis application 515 can comprise program instructions to implement logical function(s) and/or operations of the system. The program instructions may be embodied in the form of source code that comprises human-readable statements written in a programming language or machine code that comprises numerical instructions recognizable by a suitable execution system such as a processor in a computer system or other system. The machine code may be converted from the source code, etc. If embodied in hardware, each block may represent a circuit or a number of interconnected circuits to implement the specified logical function(s).

Also, any logic or application described herein, including the image acquisition application 512 and data analysis application 515 that comprises software or code can be embodied in any non-transitory computer-readable medium for use by or in connection with an instruction execution system such as, for example, a processor 503 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A portable melanoma detection device, comprising:
an imaging device; and
a processing or computing device communicatively coupled with the imaging device, the processing or computing device configured to:
receive one or more image of a skin blemish captured by the imaging device;
analyze the one or more image to determine a condition of the skin blemish, the condition indicating whether the skin blemish is malignant or benign; and
render the one or more image for display in a user interface of the processing or computing device, the one or more image displayed with the condition of the skin blemish.

2. The detection device of claim 1, wherein analysis of the one or more image is implemented by a deep learning classifier trained to determine the condition and a confidence number associated with the determined condition.

3. The detection device of claim 2, wherein a malignant condition is indicated by a confidence number that is greater than 5.

4. The detection device of claim 2, wherein the deep learning classifier determines the condition based upon averaging of analysis of a plurality of transformed versions of the one or more image.

5. The detection device of claim 4, wherein the one or more image is flipped, transposed, or both to generate the plurality of transformed versions.

6. The detection device of claim 4, wherein averaging of the analysis comprises averaging an output of the deep learning classifier associated with analysis of each of the plurality of transformed versions.

7. The detection device of claim 1, wherein acquisition of the one or more image is controlled through the user interface of the processing or computing device.

8. The detection device of claim 7, wherein the one or more image is acquired directly from the imaging device or from memory of the processing or computing device.

9. The detection device of claim 7, wherein the one or more image is acquired from memory of the processing or computing device.

10. The detection device of claim 1, wherein the skin blemish is a mole.

11. A method for melanoma detection, comprises:
acquiring an image of a skin blemish captured by an imaging device of a portable melanoma detection device;
analyzing, by a processing or computing device of the portable melanoma detection device, the image to determine a condition of the skin blemish, the condition indicating whether the skin blemish is malignant or benign; and
rendering, by a processing or computing device, the image for display in a user interface of the portable melanoma detection device, the one or more image displayed with the condition of the skin blemish.

12. The method of claim 11, comprising determining that the skin blemish is a malignant melanoma and identifying a treatment for the malignant melanoma.

13. The method of claim 12, wherein the treatment comprises removal of the malignant melanoma.

14. The method of claim 11, wherein analysis of the image is implemented by a deep learning classifier trained to determine the condition based upon a confidence number.

15. The method of claim 14, wherein the analysis of the image comprises:
generating a plurality of transformed versions by flipping the image, transposing the image, or both;
analyzing each of the plurality of transformed versions to determine corresponding confidence numbers; and
determining the condition of the skin blemish based upon an average of the corresponding confidence numbers.

16. The method of claim 14, wherein the confidence number is an output of the deep learning classifier.

17. The method of claim 14, wherein the confidence number is in a range from 1 to 10.

18. The method of claim 11, comprising:
capturing the image with the imaging device of the portable melanoma detection device; and
storing the image in memory of the portable melanoma detection device, where the image is acquired from the memory.

19. The method of claim 11, comprising capturing the image with the imaging device of the portable melanoma detection device, wherein the image is acquired directly from the imaging device after capture.

20. The method of claim 11, wherein the skin blemish is a mole.
